(19) United States
(12) Patent Application Publication
(10) Pub. No.: US 2006/0188949 A1
(43) Pub. Date: Aug. 24, 2006

(54) Use of protein PLST as a marker for colorectal cancer

(75) Inventors: Michael Tacke, Muenchen (DE); Peter Berndt, Basel (CH); Marie-Luise Hagmann, Penzberg (DE); Johann Karl, Peissenberg (DE); Hanno Langen, Steinen (DE); Stefan Palme, Penzberg (DE); Markus Roessler, Germering (DE); Wolfgang Rollinger, Polling (DE); Werner Zolg, Weilheim-Unterhausen (DE)

Correspondence Address: Roche Diagnostics Corporation, Inc., 9115 Hague Road, PO Box 50457, Indianapolis, IN, 46250-0457, US

(73) Assignee: Roche Diagnostics Operations, Inc., Indianapolis, IN (US)

(21) Appl. No.: 11/348,841

(22) Filed: Feb. 7, 2006

Related U.S. Application Data
(63) Continuation of application No. PCT/EP04/08866, filed on Aug. 6, 2004.

(30) Foreign Application Priority Data
Aug. 8, 2003 (EP) EP 03017580.6

(51) Int. Cl. G01N 33/574

(52) U.S. Cl. 435/7.23

(57) ABSTRACT

The present invention relates to the diagnosis of colorectal cancer. It discloses the use of protein PLST (T-plastin) in the diagnosis of colorectal cancer. It relates to a method for diagnosis of colorectal cancer from a liquid sample, derived from an individual by measuring PLST in said sample. Measurement of PLST can, e.g., be used in the early detection or diagnosis of colorectal cancer.

### RELATED APPLICATIONS

This application is a continuation of PCT/EP2004/008866 filed Aug. 6, 2004, and claims priority to European application EP 03017580.6 filed Aug. 8, 2003.

### FIELD OF THE INVENTION

The present invention relates to the diagnosis of colorectal cancer. It discloses the use of the protein T-plastin (PLST) in the diagnosis of colorectal cancer. Furthermore, it especially relates to a method for diagnosis of colorectal cancer from a liquid sample, derived from an individual by measuring PLST in said sample. Measurement of PLST can, e.g., be used in the early detection or diagnosis of colorectal cancer.

### BACKGROUND OF THE INVENTION

Cancer remains a major public health challenge despite progress in detection and therapy. Amongst the various types of cancer, colorectal cancer (CRC) is one of the most frequent cancers in the Western world.

The earlier cancer can be detected/diagnosed, the better is the overall survival rate. This is especially true for CRC. The prognosis in advanced stages of tumor is poor. More than one third of the patients will die from progressive disease within five years after diagnosis, corresponding to a survival rate of about 40% for five years. Current treatment is only curing a fraction of the patients and clearly has the best effect on those patients diagnosed in an early stage of disease.

With regard to CRC as a public health problem, it is essential that more effective screening and preventative measures for colorectal cancer be developed.

The earliest detection procedures available at present for colorectal cancer involve using tests for fecal blood or endoscopic procedures. However, significant-tumor size must typically exist before fecal blood is detected. The sensitivity of the guaiac-based fecal occult blood tests is ˜26%, which means 74% of patients with malignant lesions will remain undetected (Ahlquist, D. A., Gastroenterol. Clin. North Am. 26 (1997) 41-55). The visualization of precancerous and cancerous lesions represents the best approach to early detection, but colonoscopy is invasive with significant costs, risks, and complications (Silvis, S. E., et al., JAMA 235 (1976) 928-930; Geenen, J. E., et al., Am. J. Dig. Dis. 20 (1975) 231-235; Anderson, W. F., et al., J. Natl. Cancer Institute 94 (2002) 1126-1133).

In the recent years a tremendous amount of so-called colon specific or even so-called colorectal cancer specific genes has been reported. The vast majority of the corresponding research papers or patent applications are based on data obtained by analysis of RNA expression patterns in colon (cancer) tissue versus a different tissue or an adjacent normal tissue, respectively. Such approaches may be summarized as differential mRNA display techniques.

As an example for data available from mRNA-display techniques, WO 01/96390 shall be mentioned and discussed. This application describes and claims more than two hundred isolated polynucleotides and the corresponding polypeptides as such, as well as their use in the detection of CRC. However, it is general knowledge that differences on the level of mRNA are not mirrored by the level of the corresponding proteins. A protein encoded by a rare mRNA may be found in very high amounts and a protein encoded by an abundant mRNA may nonetheless be hard to detect and find at all. This lack of correlation between mRNA-level and protein level is due to reasons like mRNA stability, efficiency of translation, stability of the protein, etc.

There also are recent approaches investigating the differences in protein patterns between different tissues or between healthy and diseased tissue in order to identify candidate marker molecules which might be used in the diagnosis of CRC. Brünagel, G., et al., Cancer Research 62 (2002) 2437-2442 have identified seven nuclear matrix proteins which appear to be more abundant in CRC tissue as compared to adjacent normal tissue. No data from liquid samples obtained from an individual are reported.

WO 02/078636 reports about nine colorectal cancer-associated spots as found by surface-enhanced laser desorption and ionization (SELDI). These spots are seen more frequently in sera obtained from patients with CRC as compared to sera obtained from healthy controls. However, the identity of the molecule(s) comprised in such spot, e.g., its (their sequence), is not known.

Despite the large and ever growing list of candidate protein markers in the field of CRC, to date clinical/diagnostic utility of these molecules is not known. In order to be of clinical utility a new diagnostic marker as a single marker should be at least as good as the best single marker known in the art. Or, a new marker should lead to a progress in diagnostic sensitivity and/or specificity either if used alone or in combination with one or more other markers, respectively. The diagnostic sensitivity and/or specificity of a test is best assessed by its receiver-operating characteristics, which will be described in detail below.

At present, only diagnostic blood tests based on the detection of carcinoembryonic antigen (CEA), a tumor-associated glycoprotein, are available to assist diagnosis in the field of CRC. CEA is increased in 95% of tissue samples obtained from patients with colorectal, gastric, and pancreatic cancers and in the majority of breast, lung, and head and neck carcinomas (Goldenberg, D. M., et al., J. Natl. Cancer Inst. (Bethesda) 57 (1976) 11-22). Elevated CEA levels have also been reported in patients with nonmalignant disease, and many patients with colorectal cancer have normal CEA levels in the serum, especially during the early stage of the disease (Carriquiry, L. A., and Pineyro, A., Dis. Colon Rectum 42 (1999) 921-929; Herrera, M. A., et al., Ann. Surg. 183 (1976) 5-9; Wanebo, H. J., et al., N. Engl. J. Med. 299 (1978) 448-451). The utility of CEA as measured from serum or plasma in detecting recurrences is reportedly controversial and has yet to be widely applied (Martell, R. E., et al., Int. J. Biol. Markers 13 (1998) 145-149; Moertel, C. G., et al., JAMA 270 (1993) 943-947).

In light of the available data, serum CEA determination possesses neither the sensitivity nor the specificity to enable its use as a screening test for colorectal cancer in the asymptomatic population (Reynoso, G., et al., JAMA 220 (1972) 361-365; Sturgeon, C., Clinical Chemistry 48 (2002) 1151-1159).

Whole blood, serum or plasma are the most widely used sources of sample in clinical routine. The identification of an early CRC tumor marker that would allow reliable cancer detection or provide early prognostic information could lead to a diagnostic assay that would greatly aid in the diagnosis and in the management of this disease. Therefore, an urgent clinical need exists to improve the diagnosis of CRC from blood. It is especially important to improve the early diagnosis of CRC, since for patients diagnosed early on chances of survival are much higher as compared to those diagnosed at a progressed stage of disease.

### SUMMARY OF THE INVENTION

It was the task of the present invention to investigate whether a new marker can be identified which may aid in CRC diagnosis.

Surprisingly, it has been found that use of protein PLST can at least partially overcome the problems known from the state of the art.

The present invention therefore relates to a method for the diagnosis of colorectal cancer comprising the steps of a) providing a liquid sample obtained from an individual, b) contacting said sample with a specific binding agent for PLST under conditions appropriate for formation of a complex between said binding agent and PLST, and c) correlating the amount of complex formed in (b) to the diagnosis of colorectal cancer.

Another preferred embodiment of the invention is a method for the diagnosis of colorectal cancer comprising the steps of a) contacting a liquid sample obtained from an individual with a specific binding agent for PLST under conditions appropriate for formation of a complex between said binding agent and PLST, and b) correlating the amount of complex formed in (a) to the diagnosis of colorectal cancer.

### BRIEF DESCRIPTION OF THE DRAWINGS

FIG. 1: Identification of intact PLST with an apparent MW of 70 kDa and an isoelectric point of about pH 5.5 in colon tumor tissue. FIG. 1 shows a typical example of a 2D-gel, loaded with a tumor sample (left side), and a gel, loaded with a matched control sample (right side) obtained from adjacent healthy mucosa. The circle in the enlarged section of these gels indicates the position for the protein PLST. This protein was not detectable by the same method in healthy mucosa.

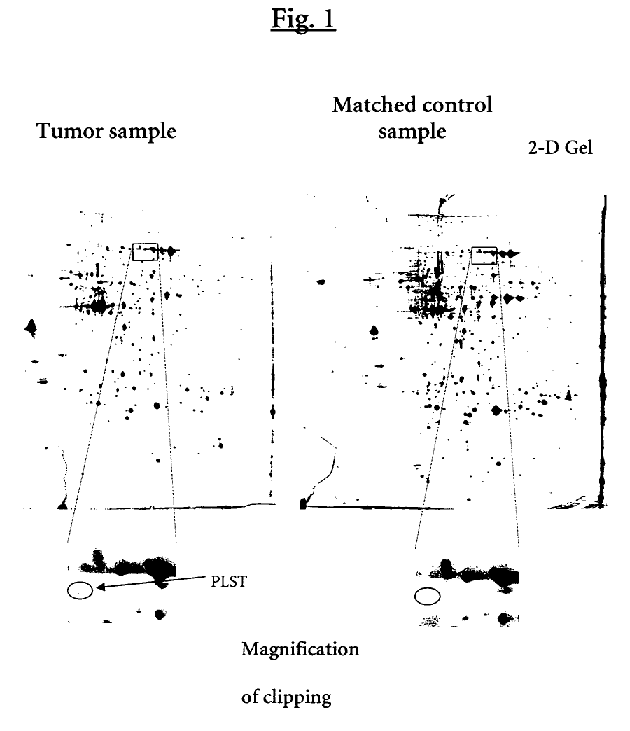

FIG. 2: Typical example of a Western-Blot. A polyacrylamide gel was loaded with tissue lysates from colorectal tumor tissue and adjacent healthy control tissue from 4 patients (subject 13: colon ca (carcinoma), Dukes B; subject 14: colon ca, Dukes B; subject 27: rectum ca, Dukes B; and subject 29: rectum ca, Dukes A) and after electrophoresis the proteins were blotted onto a nitrocellulose membrane. Presence of PLST in the samples was tested using a polyclonal rabbit anti-PLST serum. Lanes containing tumor lysates are indicated with “T”, lanes containing normal control tissue with “N”. The arrow indicates the position in the gel of the PLST band.

### DETAILED DESCRIPTION OF THE INVENTION

As the skilled artisan will appreciate, any such diagnosis is made in vitro. The patient sample is discarded afterwards. The patient sample is solely used for the in vitro diagnostic method of the invention and the material of the patient sample is not transferred back into the patient's body. Typically, the sample is a liquid sample.

The protein PLST (T-plastin; SWISS-PROT: P13797) is characterized by the sequence given SEQ ID NO: 1. The corresponding cloned human cDNA, which was isolated and characterized by Lin et al. (Lin et al. (1988), Mol. Cell. Biol. 8, 4659-4668), encodes a 70-kDa protein. Later on, Lin et al. (Lin et al. (1993), J. Biol. Chem. 268, 2781-2792) mapped the corresponding gene to the X chromosome.

Plastins are a family of actin-binding proteins that are conserved throughout eukaryote evolution. They are actin-binding proteins characterized by two actin-binding domains and two calcium-binding sites (Lin et al. (1988), Mol. Cell. Biol. 8, 4659-4668), involved in actin-bundling. In humans, two isoforms, L- and T-plastin (PLST), have been identified. The third human member is fimbrin. PLST has been found in cells of solid tissues, that have replicative potential (fibroblasts, endothelial cells, epithelial cells, etc.) (Lin et al. (1993), J. Biol. Chem. 268, 2781-2792).

While it was shown for L-plastin that an activation of the corresponding gene occurs in most human cancer cells (Park et al. (1994), Cancer Res. 54, 1775-1781) and that expression of this gene increases during colorectal cancer progression (Otsuka et al. (2001), Biochem. Biophys. Res. Commun. 289, 876-881), information towards an association of PLST and cancer is restricted to transcriptional analysis, that proved that increased levels of PLST-mRNA are found in cisplatin (a potent anticancer agent)-resistant human cancer cells (Hisano et al. (1996), FEBS Lett. 397, 101-107).

Protein levels of PLST have been assessed with immunoblotting techniques during developmental processes in the rat (Daudet and Lebart (2002), Cell. Motil. Cytoskeleton. 53, 326-336).

As obvious to the skilled artisan, the present invention shall not be construed to be limited to the full-length protein PLST of SEQ ID NO: 1. Physiological or artificial fragments of PLST, secondary modifications of PLST, as well as allelic variants of PLST are also encompassed by the present invention. Artificial fragments preferably encompass a peptide produced synthetically or by recombinant techniques, which at least comprises one epitope of diagnostic interest consisting of at least 6 contiguous amino acids as derived from the sequence disclosed in SEQ ID NO: 1. Such fragment may advantageously be used for generation of antibodies or as a standard in an immunoassay. More preferred the artificial fragment comprises at least two epitopes of interest appropriate for setting up a sandwich immunoassay.

In preferred embodiments, the novel marker PLST may be used for monitoring as well as for screening purposes.

When used in patient monitoring the diagnostic method according to the present invention may help to assess tumor load, efficacy of treatment and tumor recurrence in the follow-up of patients. Increased levels of PLST are directly correlated to tumor burden. After chemotherapy a short term (few hours to 14 days) increase in PLST may serve as an indicator of tumor cell death. In the follow-up of patients (from 3 months to 10 years) an increase of PLST can be used as an indicator for tumor recurrence.

In a preferred embodiment the diagnostic method according to the present invention is used for screening purposes. I.e., it is used to assess subjects without a prior diagnosis of CRC by measuring the level of PLST and correlating the level measured to the presence or absence of CRC.

Colorectal cancer most frequently progresses from adenomas (polyps) to malignant carcinomas. The different stages of CRC used to be classified according to Dukes' stages A to D.

The staging of cancer is the classification of the disease in terms of extent, progression, and severity. It groups cancer patients so that generalizations can be made about prognosis and the choice of therapy.

Today, the TNM system is the most widely used classification of the anatomical extent of cancer. It represents an internationally accepted, uniform staging system. There are three basic variables: T (the extent of the primary tumor), N (the status of regional lymph nodes) and M (the presence or absence of distant metastases). The TNM criteria are published by the UICC (International Union Against Cancer), Sobin, L. H., Wittekind, Ch. (eds): TNM Classification of Malignant Tumours, fifth edition, 1997.

What is especially important is, that early diagnosis of CRC translates to a much better prognosis. Malignant tumors of the colorectum arise from benign tumors, i.e. from adenoma. Therefore, best prognosis have those patients diagnosed at the adenoma stage. Patients diagnosed as early as in stage Tᵢₛ, N0, M0 or T1-3; N0; M0, if treated properly have a more than 90% chance of survival 5 years after diagnosis as compared to a 5-years survival rate of only 10% for patients diagnosed when distant metastases are already present.

In the sense of the present invention early diagnosis of CRC refers to a diagnosis at a pre-malignant state (adenoma) or at a tumor stage where no metastases at all (neither proximal nor distal), i.e., adenoma, Tᵢₛ, N0, M0 or T1-4; N0; M0 are present. Tᵢₛ denotes carcinoma in situ.

In a preferred embodiment PLST is used to diagnose CRC as early as in the adenoma stage.

It is further preferred, that CRC is diagnosed when it has not yet fully grown through the bowel wall and thus neither the visceral peritoneum is perforated nor other organs or structures are invaded, i.e., that diagnosis is made at stage Tᵢₛ, N0, M0 or T1-3; N0; M0 (=Tᵢₛ−3; N0; M0).

The diagnostic method according to the present invention is based on a liquid sample which is derived from an individual. Unlike to methods known from the art PLST is specifically measured from this liquid sample by use of a specific binding agent.

A specific binding agent is, e.g., a receptor for PLST, a lectin binding to PLST or an antibody to PLST. A specific binding agent has at least an affinity of 10⁷ l/mol for its corresponding target molecule. The specific binding agent preferably has an affinity of 10⁸ l/mol or even more preferred of 10⁹ l/mol for its target molecule. As the skilled artisan will appreciate the term specific is used to indicate that other biomolecules present in the sample do not significantly bind to with the binding agent specific for PLST. Preferably, the level of binding to a biomolecule other than the target molecule results in a binding affinity which is only 10%, more preferably only 5% of the affinity of the target molecule or less. A most preferred specific binding agent will fulfill both the above minimum criteria for affinity as well as for specificity.

A specific binding agent preferably is an antibody reactive with PLST. The term antibody refers to a polyclonal antibody, a monoclonal antibody, fragments of such antibodies, as well as to genetic constructs comprising the binding domain of an antibody.

Any antibody fragment retaining the above criteria of a specific binding agent can be used. Antibodies are generated by state of the art procedures, e.g., as described in Tijssen (Tijssen, P., Practice and theory of enzyme immunoassays 11 (1990) the whole book, especially pages 43-78; Elsevier, Amsterdam). In addition, the skilled artisan is well aware of methods based on immunosorbents that can be used for the specific isolation of antibodies. By these means the quality of polyclonal antibodies and hence their performance in immunoassays can be enhanced. (Tijssen, P., supra, pages 108-115).

For the achievements as disclosed in the present invention polyclonal antibodies raised in rabbits have been used. However, clearly also polyclonal antibodies from different species, e.g. rats or guinea pigs, as well as monoclonal antibodies can also be used. Since monoclonal antibodies can be produced in any amount required with constant properties, they represent ideal tools in development of an assay for clinical routine. The generation and use of monoclonal antibodies to PLST in a method according to the present invention is yet another preferred embodiment.

As the skilled artisan will appreciate now, that PLST has been identified as a marker which is useful in the diagnosis of CRC, alternative ways may be used to reach a result comparable to the achievements of the present invention. For example, alternative strategies to generate antibodies may be used. Such strategies comprise amongst others the use of synthetic peptides, representing an epitope of PLST for immunization. Alternatively, DNA Immunization also known as DNA vaccination may be used.

For measurement the liquid sample obtained from an individual is incubated with the specific binding agent for PLST under conditions appropriate for formation of a binding agent PLST-complex. Such conditions need not be specified, since the skilled artisan without any inventive effort can easily identify such appropriate incubation conditions.

As a final step according to the method disclosed in the present invention the amount of complex is measured and correlated to the diagnosis of CRC. As the skilled artisan will appreciate there are numerous methods to measure the amount of the specific binding agent PLST-complex all described in detail in relevant textbooks (cf., e.g., Tijssen P., supra, or Diamandis, et al., eds. (1996) Immunoassay, Academic Press, Boston).

Preferably PLST is detected in a sandwich type assay format. In such assay a first specific binding agent is used to capture PLST on the one side and a second specific binding agent, which is labeled to be directly or indirectly detectable is used on the other side.

As mentioned above, it has surprisingly been found that PLST can be measured from a liquid sample obtained from an individual sample. No tissue and no biopsy sample is required to apply the marker PLST in the diagnosis of CRC.

In a preferred embodiment the method according to the present invention is practiced with serum as liquid sample material.

In a further preferred embodiment the method according to the present invention is practiced with plasma as liquid sample material.

In a further preferred embodiment the method according to the present invention is practiced with whole blood as liquid sample material.

Furthermore stool can be prepared in various ways known to the skilled artisan to result in a liquid sample as well. Such sample liquid derived from stool also represents a preferred embodiment according to the present invention.

Whereas application of routine proteomics methods to tissue samples, leads to the identification of many potential marker candidates for the tissue selected, the inventors of the present invention have surprisingly been able to detect protein PLST in a bodily fluid sample. Even more surprising they have been able to demonstrate that the presence of PLST in such liquid sample obtained from an individual can be correlated to the diagnosis of colorectal cancer.

Antibodies to PLST with great advantage can be used in established procedures, e.g., to detect colorectal cancer cells in situ, in biopsies, or in immunohistological procedures.

Preferably, an antibody to PLST is used in a qualitative (PLST present or absent) or quantitative (PLST amount is determined) immunoassay.

Measuring the level of protein PLST has proven very advantageous in the field of CRC. Therefore, in a further preferred embodiment, the present invention relates to use of protein PLST as a marker molecule in the diagnosis of colorectal cancer from a liquid sample obtained from an individual.

The term marker molecule is used to indicate that an increased level of the analyte PLST as measured from a bodily fluid of an individual marks the presence of CRC.

It is especially preferred to use the novel marker PLST in the early diagnosis of colorectal cancer.

The use of protein PLST itself, represents a significant progress to the challenging field of CRC diagnosis. Combining measurements of PLST with other known markers, like CEA, or with other markers of CRC yet to be discovered, leads to further improvements. Therefore in a further preferred embodiment the present invention relates to the use of PLST as a marker molecule for colorectal cancer in combination with one or more marker molecules for colorectal cancer in the diagnosis of colorectal cancer from a liquid sample obtained from an individual. In this regard, the expression “one or more” denotes 1 to 10, preferably 1 to 5, more preferred 3. Preferred selected other CRC markers with which the measurement of PLST may be combined are CEA, CA 19-9, CA 724, and/or CA 242. Thus, a very much preferred embodiment of the present invention is the use of protein PLST as a marker molecule for colorectal cancer in combination with one or more marker molecules for colorectal cancer in the diagnosis of colorectal cancer from a liquid sample obtained from an individual, whereby the at least one other marker molecule is selected from the group consisting of CEA, CA 19-9, CA 724, and CA 242. Very preferred the marker PLST is used in combination with CEA.

Diagnostic reagents in the field of specific binding assays, like immunoassays, usually are best provided in the form of a kit, which comprises the specific binding agent and the auxiliary reagents required to perform the assay. The present invention therefore also relates to an immunological kit comprising at least one specific binding agent for PLST and auxiliary reagents for measurement of PLST.

Accuracy of a test is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1—specificity for the complete range of decision thresholds. On the y-axis is sensitivity, or the true-positive fraction [defined as (number of true-positive test results) (number of true-positive+number of false-negative test results)]. This has also been referred to as positivity in the presence of a disease or condition. It is calculated solely from the affected subgroup. On the x-axis is the false-positive fraction, or 1—specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

Clinical utility of the novel marker PLST has been assessed in comparison to and in combination with the established marker CEA using a receiver operator curve analysis (ROC; Zweig, M. H., and Campbell, G., Clin. Chem. 39 (1993) 561-577). This analysis has been based on well-defined patient cohorts consisting of 50 samples each from patients in T1-3; N0; M0, more progressed tumor, i.e., T4 and/or various severity of metastasis (N+ and/or M+), and healthy controls, respectively.

The following examples, references, sequence listing and figures are provided to aid the understanding of the present invention, the true scope of which is set forth in the appended claims. It is understood that modifications can be made in the procedures set forth without departing from the spirit of the invention.

Abbreviations

ABTS 2,2′-azino-di-[3-ethylbenzthiazoline sulfonate (6)]diammonium salt

BSA bovine serum albumin

cDNA complementary DNA

CHAPS (3-[(3-cholamidopropyl)-dimethylammonio]-1-propane-sulfonate)

DMSO dimethyl sulfoxide

DTT dithiothreitol

EDTA ethylenediamine tetraacetic acid

ELISA enzyme-linked immunosorbent assay

HRP horseradish peroxidase

IAA iodacetamid

IgG immunoglobulin G

IEF isoelectric focussing

IPG immobilized pH gradient

LDS lithium dodecyl sulfate

MALDI-TOF matrix-assisted laser desorption/ionisation-time of flight mass spectrometry

MES mesityl, 2,4,6-trimethylphenyl

OD. optical density

PAGE polyacrylamide gel electrophoresis

PBS phosphate buffered saline

PI isoelectric point

RTS rapid translation system

SDS sodium dodecyl sulfate

### SPECIFIC EMBODIMENTS

### Example 1

### Identification of PLST as a Potential Colorectal Cancer Marker

Sources of Tissue

In order to identify tumor-specific proteins as potential diagnostic markers for colorectal cancer, analysis of three different kinds of tissue using proteomics methods is performed.

In total, tissue specimen from 10 patients suffering from colorectal cancer are analyzed. From each patient three different tissue types are collected from therapeutic resections: tumor tissue (>80% tumor) (T), adjacent healthy tissue (N) and stripped mucosa from adjacent healthy mucosa (M). The latter two tissue types serve as matched healthy control samples. Tissues are immediately snap frozen after resection and stored at −80° C. before processing. Tumors are diagnosed by histopathological criteria.

Tissue Preparation

0.8-1.2 g of frozen tissue are put into a mortar and completely frozen by liquid nitrogen. The tissue is pulverized in the mortar, dissolved in the 10-fold volume (w/v) of lysis buffer (40 mM Na-citrate, 5 mM MgCl₂, 1% Genapol X-080, 0.02% Na-azide, Complete® EDTA-free [Roche Diagnostics GmbH, Mannheim, Germany, Cat. No. 1 873 580]) and subsequently homogenized in a Wheaton® glass homogenizer (20× loose fitting, 20× tight fitting). 3 ml of the homogenate are subjected to a sucrose-density centrifugation (10-60% sucrose) for 1 h at 4500×g. After this centrifugation step three fractions are obtained. The fraction on top of the gradient contains the soluble proteins and is used for further analysis.

Isoelectric Focussing (IEF) and SDS-PAGE

For IEF, 3 ml of the suspension are mixed with 12 ml sample buffer (7 M urea, 2 M thiourea, 2% CHAPS, 0.4% IPG buffer pH 4-7, 0.5% DTT) and incubated for 1 h. The samples are concentrated in an Amicon® Ultra-15 device (Millipore GmbH, Schwalbach, Germany) and the protein concentration is determined using the Bio-Rade protein assay (Cat. No. 500-0006; Bio-Rad Laboratories GmbH, München, Germany) following the instructions of the supplier's manual. To a volume corresponding to 1.5 mg of protein sample buffer is added to a final volume of 350 μl. This solution is used to rehydrate IPG strips pH 4-7 (Amersham Biosciences, Freiburg, Germany) overnight. The IEF is performed using the following gradient protocol: 1.) 1 minute to 500 V; 2.) 2 h to 3,500 V; 3.) 22 h at constant 3,500 V giving rise to 82 kVh. After IEF, strips are stored at −80° C. or directly used for SDS-PAGE.

Prior to SDS-PAGE the strips are incubated in equilibration buffer (6 M urea, 50 mM Tris/HCl, pH 8.8, 30% glycerol, 2% SDS), for reduction DDT (15 min, +50 mg DTT/10 ml), and for alkylation IAA (15 min, +235 mg iodacetamide/10 ml) is added. The strips are put on 12.5% polyacrylamide gels and subjected to electrophoresis at 1 W/gel for 1 h and thereafter at 17 W/gel. Subsequently, the gels are fixed (50% methanol, 10% acetate) and stained overnight with Novex™ Colloidal Blue Staining Kit (Invitrogen, Karlsruhe, Germany, Cat No. LC6025, 45-7101).

Detection of PLST as a Potential Marker for Colorectal Cancer

Each patient is analyzed separately by image analysis with the ProteomeWeaver® software (Definiens AG, Germany, München). In addition, all spots of the gel are excised by a picking robot and the proteins present in the spots are identified by MALDI-TOF mass spectrometry (Ultraflex™ Tof/Tof, Bruker Daltonik GmbH, Bremen, Germany). For each patient, 4 gels from the tumor sample are compared with 4 gels each from adjacent normal and stripped mucosa tissue and analyzed for distinctive spots corresponding to differentially expressed proteins. By this means, protein PLST is found to be specifically expressed or strongly overexpressed in tumor tissue and not detectable or less strongly expressed in healthy control tissue. It therefore—amongst many other proteins—qualifies as a candidate marker for use in the diagnosis of colorectal cancer.

### Example 2

### Generation of Antibodies to the Colorectal Cancer Marker Protein PLST

Polyclonal antibody to the colorectal cancer marker protein PLST is generated for further use of the antibody in the measurement of serum and plasma and blood levels of PLST by immunodetection assays, e.g. Western Blotting and ELISA.

Recombinant Protein Expression in E. coli

In order to generate antibodies to PLST, recombinant expression of the protein is performed for obtaining immunogens. The expression is done applying a combination of the RTS 100 expression system and E. coli. In a first step, the DNA sequence is analyzed and recommendations for high yield cDNA silent mutational variants and respective PCR-primer sequences are obtained using the “ProteoExpert RTS E. coli HY” system. This is a commercial web based service (www.proteoexpert.com). Using the recommended primer pairs, the “RTS 100 E. coli Linear Template Generation Set, His-tag” (Roche Diagnostics GmbH, Mannheim, Germany, Cat. No. 3186237) system to generate linear PCR templates from the cDNA and for in-vitro transcription and expression of the nucleotide sequence coding for the PLST protein is used. For Western-blot detection and later purification, the expressed protein contains a His-tag. The best expressing variant is identified. All steps from PCR to expression and detection are carried out according to the instructions of the manufacturer. The respective PCR product, containing all necessary T7 regulatory regions (promoter, ribosomal binding site and T7 terminator) is cloned into the pBAD TOPO® vector (Invitrogen, Karlsruhe, Germany, Cat. No. K 4300/01) following the manufacturer's instructions. For expression using the T7 regulatory sequences, the construct is transformed into E. coli BL 21 (DE 3) (Studier, F. W., et al., Methods Enzymol. 185 (1990) 60-89) and the transformed bacteria are cultivated in a 1 l batch for protein expression.

Purification of His-PLST fusion protein is done following standard procedures on a Ni-chelate column. Briefly, 1 l of bacteria culture containing the expression vector for the His-PLST fusion protein is pelleted by centrifugation. The cell pellet is resuspended in lysis buffer, containing phosphate, pH 8.0, 7 M guanidium chloride, imidazole and thioglycerole, followed by homogenization using a Ultra-Turrax®. Insoluble material is pelleted by high speed centrifugation and the supernatant is applied to a Ni-chelate chromatographic column. The column is washed with several bed volumes of lysis buffer followed by washes with buffer, containing phosphate, pH 8.0 and Urea. Finally, bound antigen is eluted using a phosphate buffer containing SDS under acid conditions.

Production of Monoclonal Antibodies Against the PLST

a) Immunization of Mice

12 week old A/J mice are initially immunized intraperitoneally with 100 μg PLST. This is followed after 6 weeks by two further intraperitoneal immunizations at monthly intervals. In this process each mouse is administered 100 μg PLST adsorbed to aluminum hydroxide and 10⁹ germs of Bordetella pertussis. Subsequently the last two immunizations are carried out intravenously on the 3rd and 2nd day before fusion using 100 μg PLST in PBS buffer for each.

b) Fusion and Cloning

Spleen cells of the mice immunized according to a) are fused with myeloma cells according to Galfre, G., and Milstein, C., Methods in Enzymology 73 (1981) 3-46. In this process ca. 1*10⁸ spleen cells of the immunized mouse are mixed with 2×10⁷ myeloma cells (P3×63-Ag8-653, ATCC CRL1580) and centrifuged (10 min at 300×g and 4° C.). The cells are then washed once with RPMI 1640 medium without fetal calf serum (FCS) and centrifuged again at 400×g in a 50 ml conical tube. The supernatant is discarded, the cell sediment is gently loosened by tapping, 1 ml PEG (molecular weight 4000, Merck, Darmstadt) is added and mixed by pipetting. After 1 min in a water-bath at 37° C., 5 ml RPMI 1640 without FCS is added drop-wise at room temperature within a period of 4-5 min. Afterwards 5 ml RPMI 1640 containing 10% FCS is added drop-wise within ca. 1 min, mixed thoroughly, filled to 50 ml with medium (RPMI 1640+10% FCS) and subsequently centrifuged for 10 min at 400×g and 4° C. The sedimented cells are taken up in RPMI 1640 medium containing 10% FCS and sown in hypoxanthine-azaserine selection medium (100 mmol/l hypoxanthine, 1 μg/ml azaserine in RPMI 1640+10% FCS). Interleukin 6 at 100 U/ml is added to the medium as a growth factor.

After ca. 10 days the primary cultures are tested for specific antibody. PLST-positive primary cultures are cloned in 96-well cell culture plates by means of a fluorescence activated cell sorter. In this process again interleukin 6 at 100 U/ml is added to the medium as a growth additive.

c) Immunoglobulin Isolation from the Cell Culture Supernatants

The hybridoma cells obtained are sown at a density of 1×10⁵ cells per ml in RPMI 1640 medium containing 10% FCS and proliferated for 7 days in a fermenter (Thermodux Co., Wertheim/Main, Model MCS-104XL, Order No. 144-050). On average concentrations of 100 μg monoclonal antibody per ml are obtained in the culture supernatant. Purification of this antibody from the culture supernatant is carried out by conventional methods in protein chemistry (e.g. according to Bruck, C., et al., Methods in Enzymology 121 (1986) 587-695).

Generation of Polyclonal Antibodies

a) Immunization

For immunization, a fresh emulsion of the protein solution (100 μg/ml protein PLST) and complete Freund's adjuvant at the ratio of 1:1 is prepared. Each rabbit is immunized with 1 ml of the emulsion at days 1, 7, 14 and 30, 60 and 90. Blood is drawn and resulting anti-PLST serum used for further experiments as described in examples 3 and 4.

b) Purification of IgG (Immunoglobulin G) from Rabbit Serum by Sequential Precipitation with Caprylic Acid and Ammonium Sulfate

One volume of rabbit serum is diluted with 4 volumes of acetate buffer (60 mM, pH 4.0). The pH is adjusted to 4.5 with 2 M Tris-base. Caprylic acid (25 μl/ml of diluted sample) is added drop-wise under vigorous stirring. After 30 min the sample is centrifuged (13,000×g, 30 min, 4° C.), the pellet discarded and the supernatant collected. The pH of the supernatant is adjusted to 7.5 by the addition of 2 M Tris-base and filtered (0.2 μm).

The immunoglobulin in the supernatant is precipitated under vigorous stirring by the drop-wise addition of a 4 M ammonium sulfate solution to a final concentration of 2 M. The precipitated immunoglobulins are collected by centrifugation (8,000×g, 15 min, 4° C.).

The supernatant is discarded. The pellet is dissolved in 10 mM NaH₂PO₄/NaOH, pH 7.5, 30 mM NaCl and exhaustively dialyzed. The dialysate is centrifuged (13,000×g, 15 min, 4° C.) and filtered (0.2 μm).

Biotinylation of Polyclonal Rabbit IgG

Polyclonal rabbit IgG is brought to 10 mg/ml in 10 mM NaH₂PO₄/NaOH, pH 7.5, 30 mM NaCl. Per ml IgG solution 50 μl Biotin —N-hydroxysuccinimide (3.6 mg/ml in DMSO) are added. After 30 min at room temperature, the sample is chromatographed on Superdex 200 (10 mM NaH₂PO₄/NaOH, pH 7.5, 30 mM NaCl). The fraction containing biotinylated IgG are collected. Monoclonal antibodies are biotinylated according to the same procedure.

Digoxygenylation of Polyclonal Rabbit IgG

Polyclonal rabbit IgG is brought to 10 mg/ml in 10 mM NaH₂PO₄/NaOH, 30 mM NaCl, pH 7.5. Per ml IgG solution 50 μl digoxigenin-3-O-methylcarbonyl-ε-aminocaproic acid-N-hydroxysuccinimide ester (Roche Diagnostics, Mannheim, Germany, Cat. No. 1 333 054) (3.8 mg/ml in DMSO) are added. After 30 min at room temperature, the sample is chromatographed on Superdex® 200 (10 mM NaH₂PO₄/NaOH, pH 7.5, 30 mM NaCl). The fractions containing digoxigenylated IgG are collected. Monoclonal antibodies are labeled with digoxigenin according to the same procedure.

### Example 3

### Western Blotting for the Detection of PLST in Human Colorectal Cancer Tissue Using Polyclonal Antibody as Generated in Example 2

Tissue lysates from tumor samples and healthy control samples are prepared as described in Example 1, “Tissue preparation”.

SDS-PAGE and Western-Blotting are carried out using reagents and equipment of Invitrogen, Karlsruhe, Germany. For each tissue sample tested, 10 μg of tissue lysate are diluted in reducing NuPAGE® (Invitrogen) SDS sample buffer and heated for 10 min at 95° C. Samples are run on 4-12% NuPAGE® gels (Tris-Glycine) in the MES running buffer system. The gel-separated protein mixture is blotted onto nitrocellulose membranes using the Invitrogen XCell II™ Blot Module (Invitrogen) and the NuPAGE® transfer buffer system. The membranes are washed 3 times in PBS/0.05% Tween-20 and blocked with Roti®-Block blocking buffer (A 151.1; Carl Roth GmbH, Karlsruhe, Germany) for 2 h. The primary antibody, polyclonal rabbit anti-PLST serum (generation described in Example 2), is diluted 1:10,000 in Roti®-Block blocking buffer and incubated with the membrane for 1 h. The membranes are washed 6 times in PBS/0.05% Tween-20. The specifically bound primary rabbit antibody is labeled with a POD-conjugated polyclonal sheep anti-rabbit IgG antibody, diluted to 10 mU/ml in 0.5× Roti®-Block blocking buffer. After incubation for 1 h, the membranes are washed 6 times in PBS/0.05% Tween-20. For detection of the bound POD-conjugated anti-rabbit antibody, the membrane is incubated with the Lumi-Light^{PLUS} Western Blotting Substrate (Order-No. 2015196, Roche Diagnostics GmbH, Mannheim, Germany) and exposed to an autoradiographic film.

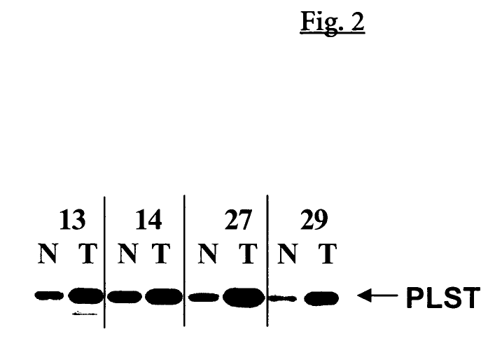

Results of a typical experiment are shown in FIG. 2. All tumor samples show a strong signal at the position of PLST, whereas only a weak signal can be detected in the lysates from adjacent normal control tissue.

### Example 4

### ELISA for the Measurement of PLST in Human Serum and Plasma Samples

For detection of PLST in human serum or plasma, a sandwich ELISA is developed. For capture and detection of the antigen, aliquots of the anti-PLST polyclonal antibody (see Example 2) are conjugated with biotin and digoxygenin, respectively.

Streptavidin-coated 96-well microtiter plates are incubated with 100 μl biotinylated anti-PLST polyclonal antibody for 60 min at 10 μg/ml in 10 mM phosphate, pH 7.4, 1% BSA, 0.9% NaCl and 0.1% Tween-20. After incubation, plates are washed three times with 0.9% NaCl, 0.1% Tween-20. Wells are then incubated for 2 h with either a serial dilution of the recombinant protein (see Example 2) as standard antigen or with diluted plasma samples from patients. After binding of PLST, plates are washed three times with 0.9% NaCl, 0.1% Tween-20. For specific detection of bound PLST, wells are incubated with 100 μl of digoxygenylated anti-PLST polyclonal antibody for 60 min at 10 μg/ml in 10 mM phosphate, pH 7.4, 1% BSA, 0.9% NaCl and 0.1% Tween-20. Thereafter, plates are washed three times to remove unbound antibody. In a next step, wells are incubated with 20 mU/ml anti-digoxigenin-POD conjugates (Roche Diagnostics GmbH, Mannheim, Germany, Catalog No. 1633716) for 60 min in 10 mM phosphate, pH 7.4, 1% BSA, 0.9% NaCl and 0.1% Tween-20. Plates are subsequently washed three times with the same buffer. For detection of antigen-antibody complexes, wells are incubated with 100 μl ABTS solution (Roche Diagnostics GmbH, Mannheim, Germany, Catalog No. 11685767) and OD is measured after 30-60 min at 405 nm with an ELISA reader.

### Example 5

### ROC Analysis to Assess Clinical Utility in Terms of Diagnostic Accuracy

Accuracy is assessed by analyzing individual liquid samples obtained from well-characterized patient cohorts, i.e., 50 patients having undergone colonoscopy and found to be free of adenoma or CRC, 50 patients diagnosed and staged as T1-3, N0, M0 of CRC, and 50 patients diagnosed with progressed CRC, having at least tumor infiltration in at least one proximal lymph node or more severe forms of metastasis, respectively. CEA as measured by a commercially available assay (Roche Diagnostics, CEA-assay (Cat. No. 1 173 1629 for Elecsys® Systems immunoassay analyzer) and PLST measured as described above are quantified in a serum obtained from each of these individuals. ROC-analysis is performed according to Zweig, M. H., and Campbell, supra. Discriminatory power for differentiating patients in the group Tᵢₛ−3, N0, M0 from healthy individuals for the combination of PLST with the established marker CEA is calculated by regularized discriminant analysis (Friedman, J. H., Regularized Discriminant Analysis, Journal of the American Statistical Association 84 (1989) 165-175).

Preliminary data indicate that PLST may also be very helpful in the follow-up of patients after surgery.

What is claimed is:

1. A method for the diagnosis of colorectal cancer comprising the steps of:
(a) providing a liquid sample obtained from an individual,
(b) contacting the sample with a specific binding agent for T-plastin (PLST) whereby a complex is formed between the binding agent and PLST,
(c) measuring an amount of complex formed, and
(d) correlating the amount of complex formed to the diagnosis of colorectal cancer.

2. The method of claim 1 wherein the sample is serum.

3. The method of claim 1 wherein the sample is plasma.

4. The method of claim 1 wherein the sample is whole blood.

5. The method of claim 1 wherein the individual is a colorectal cancer patient with an adenoma.

6. The method of claim 1 wherein the individual is a colorectal cancer patient in stage Tᵢₛ−3; N0; M0.

7. The method of claim 1 wherein step (d) further includes correlating a marker selected from the group consisting of carcinoembryonic antigen (CEA), CA 19-9, CA 72-4, and CA 242 to the diagnosis of colorectal cancer.

8. An immunological kit comprising at least one specific binding agent for PLST and auxiliary reagents for measurement of PLST.

9. A method for determining the presence or amount of T-plastin (PLST) in a biological sample comprising the steps of:
(a) contacting the sample with a specific binding agent for PLST whereby a complex is formed between the binding agent and PLST,
(b) measuring the amount of complex formed in step (a), and
(c) correlating the amount measured in step (b) to the presence or amount of PLST in the sample.


SEQUENCE LISTING (1 sequence)

SEQ ID NO: 1 - protein, 627 aa, Homo sapiens [sequence omitted]